(19) United States
(12) Patent Application Publication
(10) Pub. No.: US 2026/0174342 A1
(43) Pub. Date: Jun. 25, 2026

(54) BIOLOGICAL INFORMATION MEASUREMENT DEVICE, BIOLOGICAL INFORMATION MEASUREMENT METHOD, AND RECORDING MEDIUM

(71) Applicant: OMRON HEALTHCARE Co., Ltd., Kyoto (JP)

(72) Inventors: Yuki SAKAGUCHI, Kyoto (JP); Chiaki EBISU, Kyoto (JP); Tatsuya KOBAYASHI, Kyoto (JP); Mika EZOE, Kyoto (JP); Takanobu YAMAUCHI, Kyoto (JP); Yotaro NISHIO, Kyoto (JP); Tetsuho ONO, Kyoto (JP); Iwanori NAMBA, Kyoto (JP); Kenichi HINUMA, Kyoto (JP)

(21) Appl. No.: 19/545,719

(22) Filed: Feb. 20, 2026

Related U.S. Application Data
(63) Continuation of application No. PCT/JP2025/000863, filed on Jan. 14, 2025.

(30) Foreign Application Priority Data
Mar. 13, 2024 (JP) 2024-038976

(51) Int. Cl. A61B 5/0205; A61B 5/00; A61B 5/01; A61B 5/0537; A61B 5/107; A61B 5/11; G01G 19/44

(52) U.S. Cl. CPC A61B 5/0205; A61B 5/01; A61B 5/0537; A61B 5/107; A61B 5/1102; A61B 5/7271; A61B 5/742; G01G 19/44

(57) ABSTRACT

To improve usability in measurement of biological information. A biological information measurement device with a placement surface on which a subject is able to be placed includes: a first measurement unit that performs at least one of a first measurement process of measuring a state of a sole of a foot of the subject placed on the placement surface or a second measurement process of measuring a body motion of the subject placed on the placement surface; and a second measurement unit that, based on at least one of a measurement result of the first measurement process or a measurement result of the second measurement process, selects at least one measurement item from a plurality of measurement items related to a living body of the subject and measures biological information of the subject.

### CROSS-REFERENCE TO RELATED APPLICATIONS

This application is the U.S. national stage application filed pursuant to 35 U.S.C. 365(c) and 120 as a continuation of International Patent Application No. PCT/JP2025/000863, filed Jan. 14, 2025, which application claims priority to Japanese Patent Application No. 2024-038976, filed Mar. 13, 2024, which applications are incorporated herein by reference in their entireties.

### FIELD

The present invention relates to a biological information measurement device, a biological information measurement method, and a program or recording medium.

### BACKGROUND ART

Patent Literature 1 discloses technology of automatically switching an impedance measurement mode according to a measurement state of a subject. Patent Literature 2 discloses technology of automatically determining whether a subject is a human or an animal, based on a contact state between a sole of a foot of the subject and a measurement electrode, measuring impedance according to each of the subjects, and calculating body composition data, based on the impedance. Patent Literature 3 discloses technology of measuring characteristics of a user's cardiovascular system. Patent Literature 4 discloses a visceral fat determining device that displays visceral fat information and includes a setting registration key for setting and registering unique biological information about a body and time, a personal key for calling and measuring the unique biological information about the body, and a weight dedicated key for measuring only a weight. Patent Literature 1: JP 2005-230120 A; Patent Literature 2: JP 2005-230392 A; Patent Literature 3: JP 2014-507213 A; and, Patent Literature 4: JP 2002-238870 A.

### SUMMARY OF INVENTION

### Technical Problem

A general body composition meter includes a load cell and an electrode unit that comes into contact with a human body with which a weight measurement or a bioelectrical impedance measurement is performed, and displays a result related to a body weight or various body compositions. The time spent for such measurements is several seconds. Thus, the measurement result is not largely affected by body motion of the subject, such as movement of the head for a purpose such as checking the measurement progress state displayed on the feet side during measurement. On the other hand, when a function of performing ballistocardiogram (BCG) measurement from a load cell and impedance pulse wave (IPG) measurement from an electrode and presenting cardiovascular state information to a user is added to the body composition meter, body motion that is movement of the head or arm during measurement largely affects the measurement result. Thus, as a result, the measurement time may be prolonged, or remeasurement may be required due to a measurement error occurring as a result. A series of data collection in the body composition meter proceeds simultaneously in parallel and the results of all the measurement items are displayed in order after a predetermined measurement time has elapsed in typical configurations. Thus, a considerable measurement time including the time for displaying the results is required as the number of measurement items increases. However, in a case where a body composition meter is shared by family members, a management target may be only a body weight value, and a measurement target item may be limited to a body weight. For example, when a body weight dedicated key is provided in a body composition meter as in Patent Literature 4, the body weight dedicated key needs to be added, and a key operation before measurement is cumbersome, meaning that usability is low. In addition, when only the body weight is to be measured, the subject is expected to be on the body composition meter with his or her socks on. In this case, the bioelectrical impedance of the subject cannot be measured, and a measurement error occurs, resulting in compromised usability.

The present invention has been made in view of the above circumstances, and an object thereof is to improve usability in measurement of biological information.

### Solution to Problem

An aspect of the present invention provides a biological information measurement device with a placement surface configured to allow placement of a subject, the biological information measurement device including: a first measurement unit configured to perform at least one of a first measurement process of measuring a state of a sole of a foot of the subject placed on the placement surface or a second measurement process of measuring a body motion of the subject placed on the placement surface; and a second measurement unit configured to, based on at least one of a measurement result of the first measurement process or a measurement result of the second measurement process, select at least one measurement item from a plurality of measurement items related to a living body of the subject and measure biological information of the subject. According to the biological information measurement device, at least one measurement item is selected from the plurality of measurement items related to the living body of the subject according to the state of the sole of the foot of the subject and the body motion of the subject, and the biological information of the subject is measured, and thus, usability in the measurement of the biological information is improved.

The plurality of measurement items include a measurement item for measuring a weight of the subject placed on the placement surface, and a sensor configured to measure the body motion of the subject placed on the placement surface and a sensor configured to measure the weight of the subject placed on the placement surface are configured as a common sensor. This enables cost reduction.

The plurality of measurement items include a measurement item that is measured using a sensor configured to measure the body motion of the subject placed on the placement surface. The sensor configured to measure the body motion of the subject may be a load cell. Items for which measurement is performed using the load cell include a measurement item for measuring a weight, a measurement item for measuring standing position balance, and a measurement item for measuring a cardiovascular state. The standing position balance is, for example, the balance of the subject in a standing position.

The first measurement unit performs the first measurement process and the second measurement process, and when the measurement result of the first measurement process includes a result indicating that the subject is not barefoot and includes a result indicating interruption of a stationary state of the subject, the second measurement unit selects a measurement item for measuring a weight from the plurality of measurement items and measures the biological information of the subject. Since only the weight of the subject is measured and no measurement is performed for the other measurement items, the time from the start to the completion of the measurement of the biological information of the subject is shortened.

The first measurement unit performs the first measurement process and the second measurement process, and when the measurement result of the first measurement process includes a result indicating that the subject is barefoot and includes a result indicating interruption of a stationary state of the subject, the second measurement unit selects at least one measurement item that is measurable in a state where the subject is not still from the plurality of measurement items and measures the biological information of the subject. Examples of the measurement item that can be measured in a state where the subject is not stationary include a measurement item for measuring the weight and a measurement item for measuring the body composition for example. Since the weight and the body composition of the subject are measured and no measurement is performed for the other measurement items, the time from the start to the completion of the measurement of the biological information of the subject is shortened.

The first measurement unit performs the first measurement process and the second measurement process, and when the measurement result of the first measurement process includes a result indicating that the subject is not barefoot and includes a result indicating that the subject is continuously in a stationary state, the second measurement unit selects at least one measurement item that is measurable without using a bioelectrical impedance measurement result from the plurality of measurement items and measures the biological information of the subject. The measurement items that can be measured without using the bioelectrical impedance measurement result are the measurement item for measuring the weight and the measurement item for measuring the standing position balance. Since the weight and the standing position balance of the subject are measured and no measurement is performed for the other measurement items, the time from the start to the completion of the measurement of the biological information of the subject is shortened.

The first measurement unit performs the first measurement process and the second measurement process, and when the measurement result of the first measurement process includes a result indicating that the subject is barefoot and the measurement result of the second measurement process includes a result indicating that the subject is continuously in a stationary state, the second measurement unit selects all of the plurality of measurement items and measures the biological information of the subject. When the subject is on the placement surface of the biological information measurement device while being barefoot and the subject is continuously in a stationary state, the measurement of the biological information of the subject is performed under an assumption that the subject has an intention to measure all of the plurality of measurement items.

The biological information measurement device includes a display unit configured to display information, in which the display unit does not display the information while a measurement process is performed by the second measurement unit. The motion of the subject to see the information displayed on the display unit, such as moving the head, affects the measurement of the cardiovascular state. The display unit does not display information while the measurement process is performed by the second measurement unit, and thus it is possible to suppress the body motion of the subject, and the accuracy of measurement on the cardiovascular state system is improved.

The biological information measurement device includes a temperature measurement unit configured to measure a temperature of the sole of the foot of the subject placed on the placement surface, in which when the temperature of the foot of the subject is equal to or lower than a predetermined temperature, the first measurement unit performs the second measurement process, and the second measurement unit selects at least one measurement item that is measurable without using a bioelectrical impedance measurement result from the plurality of measurement items and measures the biological information of the subject, based on the measurement result of the second measurement process. When the sole of the foot of the subject placed on the placement surface of the biological information measurement device is in a low temperature state, the measurement accuracy of the state of the sole of the foot of the subject using the plurality of electrodes is compromised, and the subject may be erroneously determined not to be barefoot although the subject is barefoot. When the temperature of the sole of the foot of the subject placed on the placement surface of the biological information measurement device is equal to or lower than the predetermined temperature, the first measurement unit does not perform the first measurement process, and thus it is possible to avoid the erroneous determination.

Note that the present invention can also be understood as a biological information measurement method in which a computer executes at least part of the above processing, a biological information measurement method including at least part of the above processing, a program or recording medium for causing a computer to execute at least part of the above processing, or a computer-readable recording medium on which such a program is non-transitorily recorded. Note that the configurations and processes described above can be combined with one another to constitute the present invention unless the combination leads to technical contradiction.

### Advantageous Effects of Invention

According to the present invention, usability in measurement of biological information can be improved.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a diagram illustrating a schematic configuration of a biological information measurement device according to an embodiment.

FIG. 2 is a block diagram illustrating a configuration of the biological information measurement device according to the embodiment.

FIG. 3 is a diagram illustrating an arrangement of a plurality of load cells.

FIG. 4 is a diagram illustrating an arrangement of the plurality of load cells and an acceleration sensor.

FIG. 5 is a block diagram illustrating a functional configuration of a control unit.

FIG. 6 is a diagram illustrating the relationship between a BCG waveform, a foot IPG waveform, and a PTT.

FIG. 7 is a flowchart illustrating a first operation of the biological information measurement device.

FIG. 8 is a diagram illustrating a schematic configuration of a biological information measurement device according to an embodiment.

FIG. 9 is a diagram illustrating an arrangement of a photoelectric sensor.

FIG. 10 is a diagram illustrating a case where the amount of light received by the photoelectric sensor is measured to determine whether a subject is barefoot.

FIG. 11 is a diagram illustrating a case where the amount of light received by the photoelectric sensor is measured to determine whether a subject is barefoot.

FIG. 12 is a diagram illustrating a case where the amount of light received by the photoelectric sensor is measured to determine whether a subject is barefoot.

FIG. 13 is a flowchart illustrating a second operation of the biological information measurement device.

FIG. 14 is a flowchart illustrating a third operation of the biological information measurement device.

FIG. 15 is a time chart of a process of measuring a sole state, a process of measuring a body motion, a process of measuring a weight, a process of measuring a body composition, a process of measuring a standing position balance, and a process of measuring a cardiovascular state.

### DESCRIPTION OF EMBODIMENTS

Embodiments will be described below with reference to the drawings. The embodiment described below is one aspect of the present application and does not limit the scope of the present application.

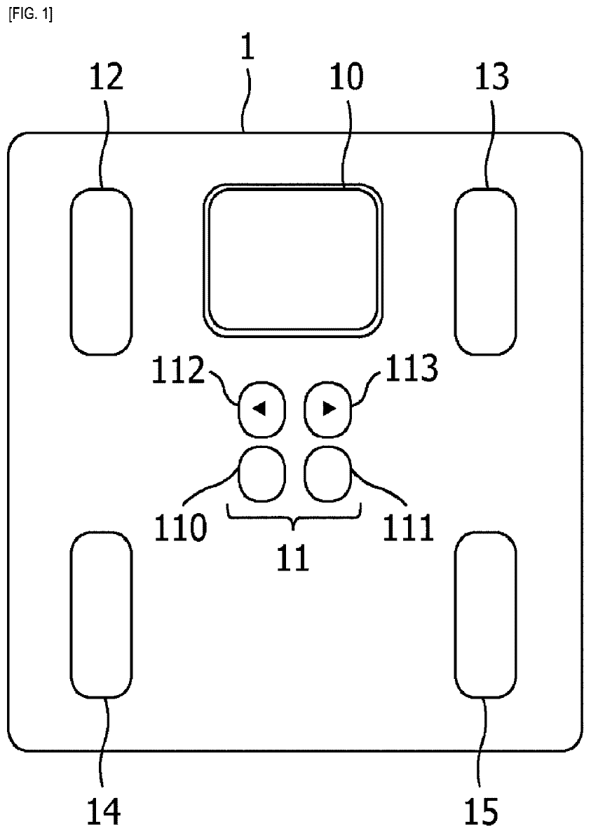

FIG. 1 is a diagram illustrating a schematic configuration of a biological information measurement device 1 according to the present embodiment. The biological information measurement device 1 includes a display unit 10, an operation unit 11, current application electrodes 12 and 13, and voltage measurement electrodes 14 and 15.

The biological information measurement device 1 has a surface (placement surface) on which a subject such as a user can be placed. The current application electrode 12 and the voltage measurement electrode 14 are arranged on the surface of the biological information measurement device 1 such that, when a subject such as a user is placed on the surface of the biological information measurement device 1, the distal end side (toe side) of the sole of the left foot comes into contact with the current application electrode 12 and the proximal end side (heel side) of the sole of the left foot comes into contact with the voltage measurement electrode 14. The current application electrode 13 and the voltage measurement electrode 15 are arranged on the surface of the biological information measurement device 1 such that, when the subject is on the surface of the biological information measurement device 1, the distal end side (toe side) of the sole of the left foot comes into contact with the current application electrode 13 and the proximal end side (heel side) of the sole of the left foot comes into contact with the voltage measurement electrode 15. The arrangement of the current application electrode 12 and the voltage measurement electrode 14 may be reversed, and the arrangement of the current application electrode 13 and the voltage measurement electrode 15 may be reversed.

The display unit 10 is provided on the surface of the biological information measurement device 1. The display unit 10 is disposed between the current application electrodes 12 and 13. The operation unit 11 is provided in a center portion of the surface of the biological information measurement device 1. The subject is placed on the surface of the biological information measurement device 1 such that the distal end side and the proximal end side of the sole of the left foot of the subject come in contact with the current application electrode 12 and the voltage measurement electrode 14, respectively, and the distal end side and the proximal end side of the sole of the right foot of the subject come in contact with the current application electrode 13 and the voltage measurement electrode 15, respectively.

The operation unit 11 includes switches 110 to 113. The switch 110 is a switch that receives input of an instruction to turn the power on or off the biological information measurement device 1 and an instruction to start or end measurement. The switch 111 is a switch that receives an instruction to display various types of data stored in the biological information measurement device 1, on the display unit 10. The switches 112 and 113 are switches that receive selection of various data displayed on the display unit 10. The switches 111 to 113 are used to receive data input.

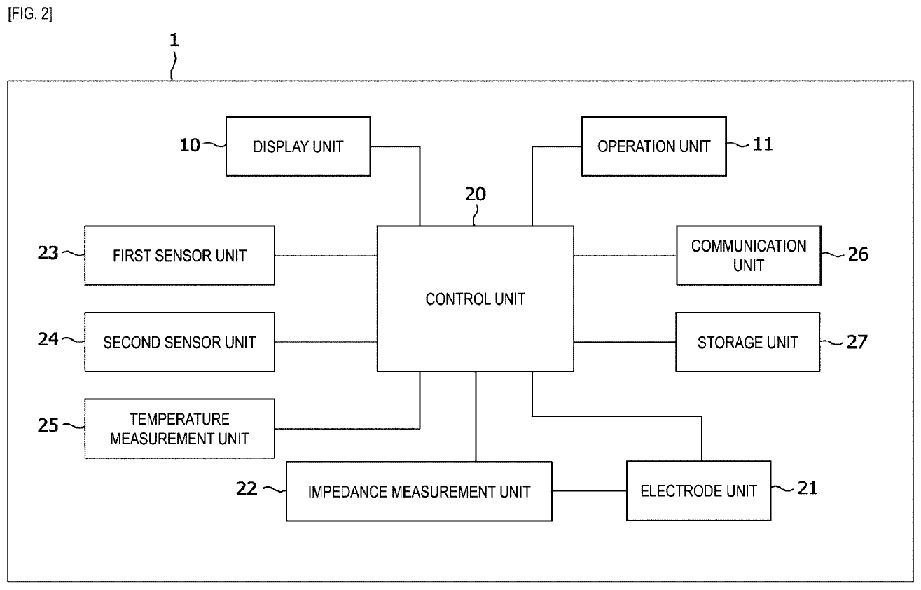

FIG. 2 is a block diagram illustrating a configuration of the biological information measurement device 1 according to the embodiment. The biological information measurement device 1 includes the display unit 10, the operation unit 11, a control unit 20, an electrode unit 21, an impedance measurement unit 22, a first sensor unit 23, a second sensor unit 24, a temperature measurement unit 25, a communication unit 26, and a storage unit 27. For example, a liquid crystal display panel such as a liquid crystal display (LCD), an electroluminescence (EL) display, or the like is used for the display unit 10. The display unit 10 displays measurement results as well as various types of information and data. The operation unit 11 receives an operation from the subject.

The control unit 20 is a control device (controller) that controls the entire operation of the biological information measurement device 1. The control unit 20 may be configured by a dedicated device or may be configured by a general-purpose computer. The control unit 20 includes hardware resources such as a central processing unit (CPU), a memory, and a storage. The memory may be a random access memory (RAM). The storage may be a non-volatile storage device such as a read only memory (ROM) or a flash memory. The functions of the control unit 20 as the processing units (functional units) are realized by loading a program stored in the storage onto a memory and executing the program by a processor. The configuration of the control unit 20 is not limited to the above. For example, the functions of the control unit 20 may be entirely or partially configured by a circuit such as an ASIC or an FPGA, or the functions of the control unit 20 may be entirely or partially executed by a cloud server or another device.

The electrode unit 21 includes the current application electrodes 12 and 13 for applying a constant current to the body of the subject to measure the bioelectrical impedance of the subject, and voltage measurement electrodes 14 and 15 for measuring a voltage under the current application. The electrode unit 21 may include other electrodes different from the current application electrodes 12 and 13 and the voltage measurement electrodes 14 and 15. The impedance measurement unit 22 measures the bioelectrical impedance of the subject, based on the values of the current applied to the body of the subject from the current application electrodes 12 and 13 and the voltage measured by the voltage measurement electrodes 14 and 15. The impedance measurement unit 22 outputs the bioelectrical impedance of the subject to the control unit 20. The impedance measurement unit 22 may output the bioelectrical impedance of the subject as an impedance pulse wave.

The first sensor unit 23 includes a photoelectric sensor. The first sensor unit 23 outputs the measurement value measured by the photoelectric sensor to the control unit 20. The second sensor unit 24 has a plurality of load cells (strain gauges). The second sensor unit 24 outputs the measurement values measured by the plurality of load cells to the control unit 20.

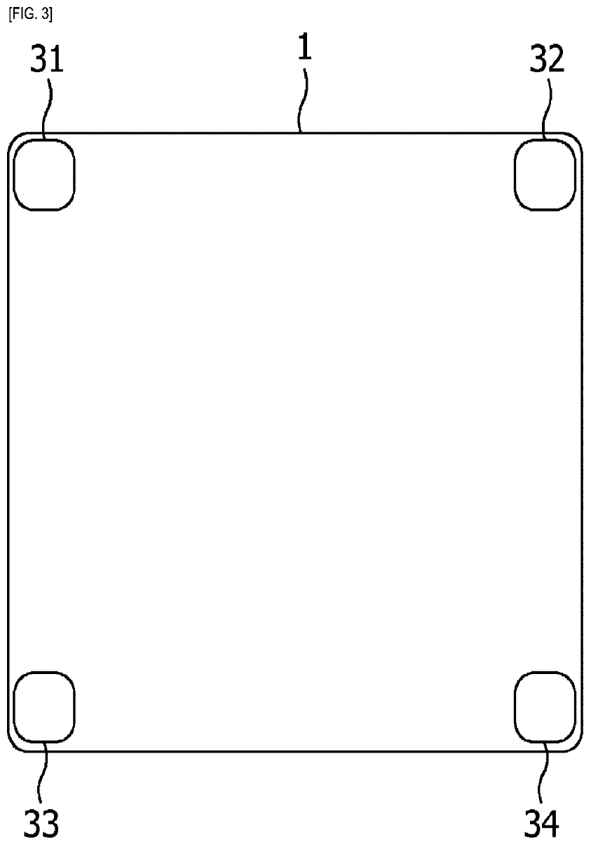

FIG. 3 is a diagram illustrating an arrangement of a plurality of load cells. As illustrated in FIG. 3, load cells 31 to 34 are disposed on the back side of the biological information measurement device 1. The load cells 31 to 34 are respectively disposed at four corners on the back side of the biological information measurement device 1. The second sensor unit 24 may include an acceleration sensor. The second sensor unit 24 may output the measurement value measured by the acceleration sensor to the control unit 20.

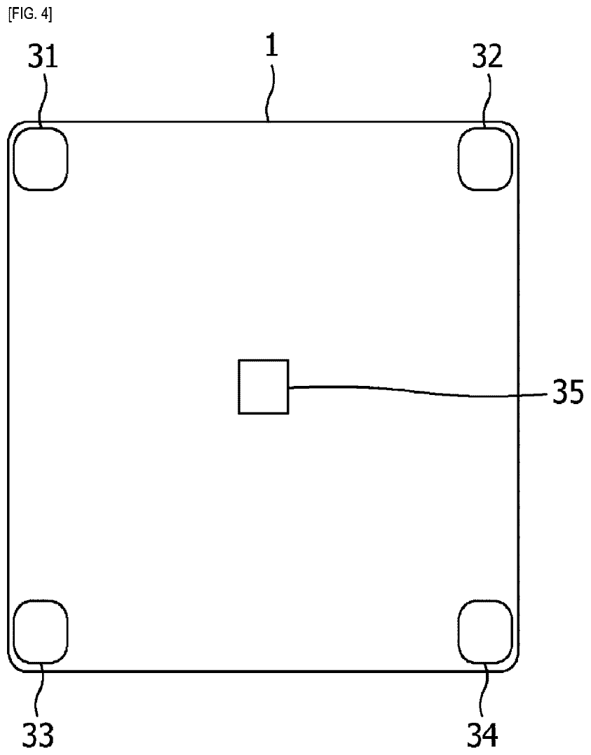

FIG. 4 is a diagram illustrating an arrangement of the plurality of load cells and an acceleration sensor. As illustrated in FIG. 4, load cells 31 to 34 and an acceleration sensor 35 are disposed on the back side of the biological information measurement device 1. The load cells 31 to 34 are respectively disposed at four corners on the back side of the biological information measurement device 1. The acceleration sensor 35 is disposed in a center portion of the back side of the biological information measurement device 1.

The temperature measurement unit 25 measures the temperature of the sole of the foot of the subject on the surface of the biological information measurement device 1. The temperature measurement unit 25 includes a temperature sensor. The temperature sensor is disposed on the surface of the biological information measurement device 1. The temperature sensor may be disposed in the vicinity of the current application electrodes 12 and 13, or may be disposed in the vicinity of the voltage measurement electrodes 14 and 15. The communication unit 26 is an interface for performing wired or wireless communication. The communication unit 26 communicates with an information processing device of the subject. The information processing device is, for example, a portable terminal, a smartphone, a tablet terminal, a personal computer, or the like.

The storage unit 27 stores various types of information and data. The storage unit 27 may include at least one of a memory such as a RAM or a storage. The storage may be a non-volatile storage device such as a ROM or a flash memory. The storage unit 27 stores various types of information such as personal data of the subject and a measurement result of the biological information measurement device 1. The personal data is used when calculating the body composition of the subject. The personal data includes at least the height and weight of the subject, and may further include the age and gender of the subject, as well as other types of information. The weight of the subject is measured when the subject gets on the surface of the biological information measurement device 1. When the subject operates the operation unit 11, the height, age, gender, and other types of information of the subject are input to the biological information measurement device 1.

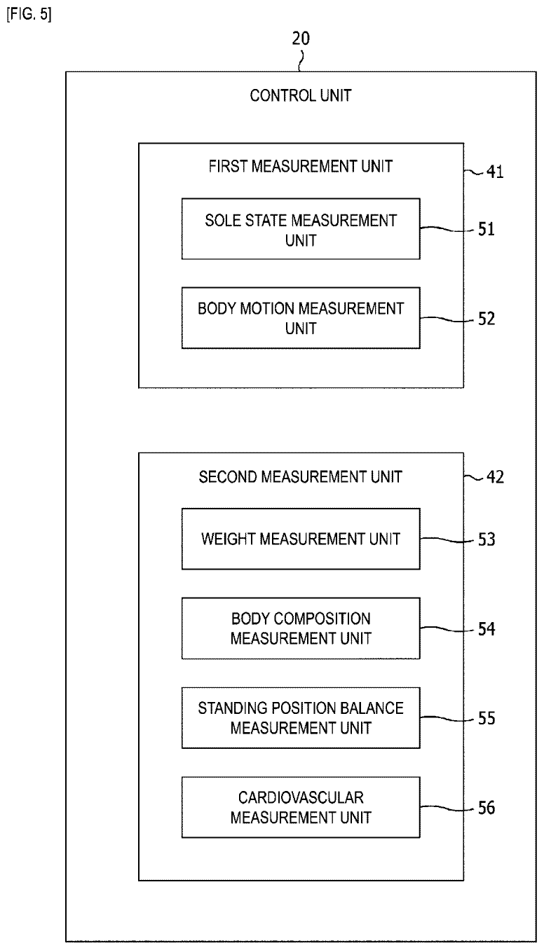

FIG. 5 is a block diagram illustrating a functional configuration of the control unit 20. The control unit 20 includes a first measurement unit 41 and a second measurement unit 42. The first measurement unit 41 includes a sole state measurement unit 51 and a body motion measurement unit 52. The second measurement unit 42 includes a weight measurement unit 53, a body composition measurement unit 54, a standing position balance measurement unit 55, and a cardiovascular measurement unit 56.

The sole state measurement unit 51 measures the state of the sole of the foot of the subject placed on the surface of the biological information measurement device 1, based on a measurement value output from at least one of the electrode unit 21 or the first sensor unit 23. The body motion measurement unit 52 measures the body motion of the subject on the surface of the biological information measurement device 1, based on the measurement value output from at least one of the first sensor unit 23, the second sensor unit 24, or the electrode unit 21. The weight measurement unit 53 measures the weight of the subject on the surface of the biological information measurement device 1, based on the measurement value output from the second sensor unit 24.

The body composition measurement unit 54 measures the body composition of the subject placed on the surface of the biological information measurement device 1, based on the personal data of the subject, the bioelectrical impedance output from the impedance measurement unit 22, and the weight of the subject according to a predetermined algorithm. The body composition is an indicator indicating a percentage or an amount of tissue that constitutes the body. Examples of the body composition include, but are not limited to, body fat percentage, visceral fat level, skeletal muscle percentage, basal metabolism, body age, BMI, muscle percentage, muscle mass, body fat mass, bone mass, water content, and the like. The personal data of the subject includes the weight and the bioelectrical impedance measured in the past. The body composition measurement unit 54 may identify the subject by comparing the weight and the bioelectrical impedance measured in the past with the weight and the bioelectrical impedance currently measured. The body composition measurement unit 54 acquires the personal data of the identified subject from the storage unit 27.

The standing position balance measurement unit 55 measures the balance of the subject in a standing position (standing position balance) on the surface of the biological information measurement device 1, based on the measurement value output from the second sensor unit 24. The standing position balance measurement unit 55 may measure the standing position balance of the subject by calculating the position of the center of gravity of the subject in the standing position. The standing position balance measurement unit 55 may measure the standing position balance of the subject according to a predetermined algorithm.

The cardiovascular measurement unit 56 measures the cardiovascular state of the subject on the surface of the biological information measurement device 1, based on the measurement value output from the second sensor unit 24 and the bioelectrical impedance output from the impedance measurement unit 22 according to a predetermined algorithm. The cardiovascular measurement unit 56 measures ballistocardiogram (BCG), which is vibration caused by the blood being pumped out from the heart, based on the measurement value output from the second sensor unit 24. The cardiovascular measurement unit 56 measures the leg impedance pulse wave (IPG) and the foot impedance pulse wave (IPG), based on the bioelectrical impedance output from the impedance measurement unit 22. The leg IPG is an indicator indicating a change in the amount of blood in the blood vessel of the leg. The foot IPG is an indicator indicating a change in the amount of blood in a blood vessel of a foot. The cardiovascular measurement unit 56 creates an average waveform of the foot IPG (foot IPG waveform) with reference to the leg IPG. The cardiovascular measurement unit 56 calculates a pulse transit time (PTT) from the BCG waveform and a feature point of the foot IPG waveform.

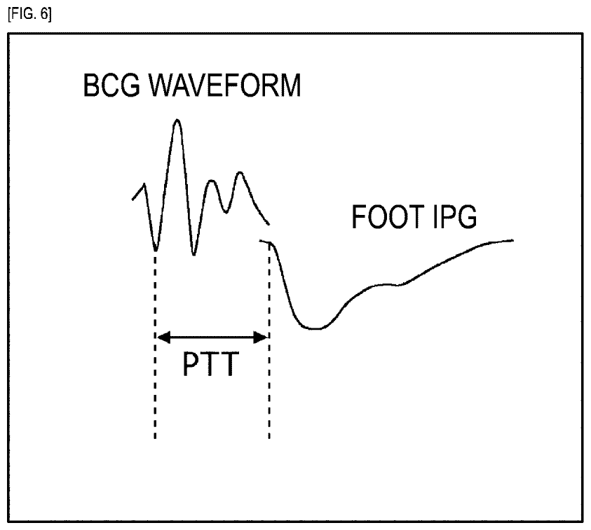

FIG. 6 is a diagram illustrating the relationship between the BCG waveform, the foot IPG waveform, and the PTT. As illustrated in FIG. 6, the pulse transit time (PTT) can be calculated by obtaining a (time) difference between a reference point of the BCG waveform and the feature point of the foot IPG waveform. The cardiovascular measurement unit 56 calculates a pulse wave velocity (PWV) by dividing a measurement section distance by the PTT. Since the value of the PWV increases as the blood vessel becomes harder, the PWV is used as an indicator indicating the hardness of the blood vessel. The cardiovascular measurement unit 56 converts the pulse wave velocity (PWV) into an indicator (cardiovascular indicator) indicating a cardiac function or a state of a blood vessel, based on a predetermined algorithm, and calculates the cardiovascular indicator. In order to measure the cardiovascular state of the subject, it is necessary to maintain for about 20 to 30 seconds, a state in which there is no body motion of the subject or a state in which the body motion of the subject is small. Therefore, the cardiovascular measurement unit 56 starts measuring the cardiovascular state of the subject with the subject making no body motion or making a small body motion.

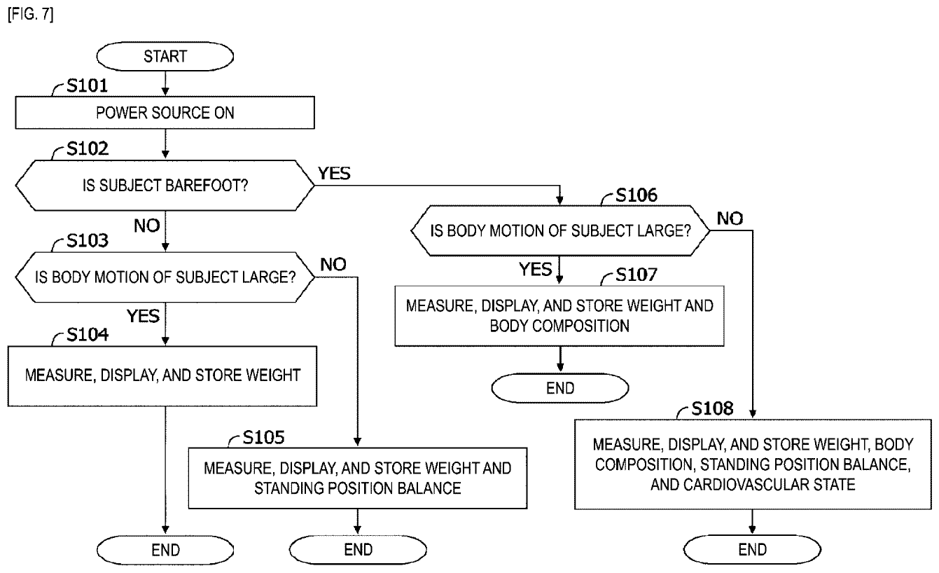

A first operation of the biological information measurement device 1 will be described with reference to FIG. 7. FIG. 7 is a flowchart illustrating the first operation of the biological information measurement device 1. For example, when a first operation mode is set for the biological information measurement device 1, the control unit 20 reads out the program stored in the storage unit 27, and thereby, each process in the flowchart of FIG. 7 is executed.

In step S101, the subject presses the switch 110 of the biological information measurement device 1 to turn on the power of the biological information measurement device 1. The power of the biological information measurement device 1 may be turned on when the subject is gets on the surface of the biological information measurement device 1 in step S101.

In step S102, the sole state measurement unit 51 measures the state of the sole of the foot of the subject placed on the surface of the biological information measurement device 1, based on a measurement value output from at least one of the electrode unit 21 or the first sensor unit 23, and whether the subject is barefoot is determined. When the subject is not barefoot (step S102; NO), the process proceeds to step S103. When the subject is barefoot (step S102; YES), the process proceeds to step S106.

The process of determining whether the subject is barefoot will be described. The process of determining whether the subject is barefoot may be performed by disposing a plurality of electrodes on each of the front side and the rear side of the surface of the biological information measurement device 1 and measuring the resistance value between the electrodes. The electrode unit 21 may include a plurality of electrodes. The front side of the surface of the biological information measurement device 1 is the toe side of the foot of the subject when the foot of the subject is placed on the surface of the biological information measurement device 1. The rear side of the surface of the biological information measurement device 1 is the heel side of the foot of the subject when the foot of the subject is placed on the surface of the biological information measurement device 1. When the subject is wearing socks, there is no difference between a resistance value between the two electrodes disposed on the front side of the surface of the biological information measurement device 1 and a resistance value between the two electrodes disposed on the rear side of the surface of the biological information measurement device 1. The sole state measurement unit 51 may perform the process of determining whether the subject is barefoot by measuring the resistance value between two electrodes disposed on the front side of the surface of the biological information measurement device 1 and the resistance value between two electrodes disposed on the rear side of the surface of the biological information measurement device 1. One of the two electrodes disposed on the front side of the surface of the biological information measurement device 1 may be the current application electrode 12 or 13. One of the two electrodes disposed on the rear side of the surface of biological information measurement device 1 may be the voltage measurement electrode 14 or 15.

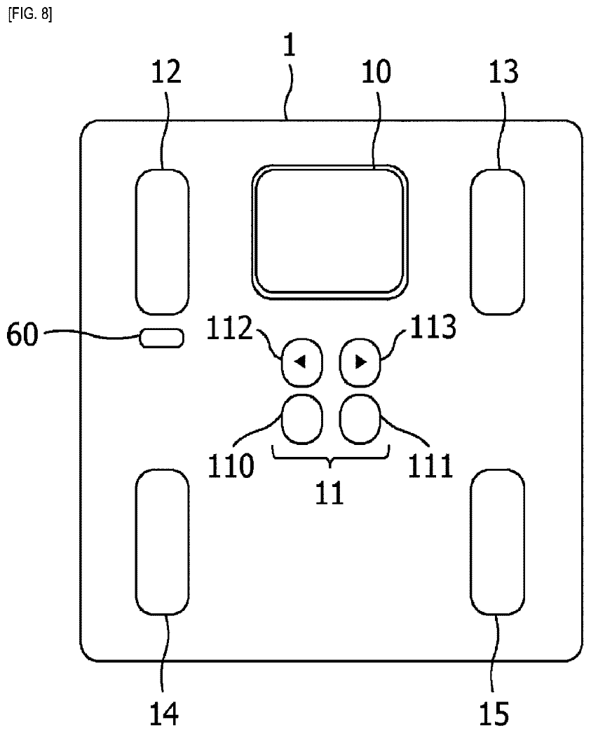

FIG. 8 is a diagram illustrating a schematic configuration of a biological information measurement device 1 according to the present embodiment. As illustrated in FIG. 8, an auxiliary electrode 60 may be disposed on the front side of the surface of the biological information measurement device 1. The sole state measurement unit 51 may perform the process of determining whether the subject is barefoot by measuring a resistance value between the current application electrode 12 and the auxiliary electrode 60 and a resistance value between the voltage measurement electrode 14 and the auxiliary electrode 60. The auxiliary electrode 60 may be disposed not only at the position illustrated in FIG. 8 but also at any position between the current application electrode 12 and the voltage measurement electrode 14. The auxiliary electrode 60 may be disposed at any position between the current application electrode 13 and the voltage measurement electrode 15. Since the plantar arch portion of the sole of the foot of the subject is unlikely to come into contact with the auxiliary electrode 60, it is not preferable to dispose the auxiliary electrode 60 at a location corresponding to the plantar arch portion of the sole of the foot of the subject. In general, the heel side of the foot of the subject is stiffer than the toe side of the foot of the subject, and the difference between the resistance value of the toe side of the foot of the subject and the resistance value of the heel side of the foot of the subject is large. On the other hand, when the subject is wearing footwear such as socks, the difference between the resistance value on the toe side of the foot of the subject and the resistance value on the heel side of the foot of the subject is small. When the difference (difference value) between the resistance value on the toe side of the foot of the subject and the resistance value on the heel side of the foot of the subject is equal to or greater than the threshold, the sole state measurement unit 51 determines that the subject is barefoot. When the difference (difference value) between the resistance value on the toe side of the foot of the subject and the resistance value on the heel side of the foot of the subject is less than the threshold, the sole state measurement unit 51 determines that the subject is not barefoot.

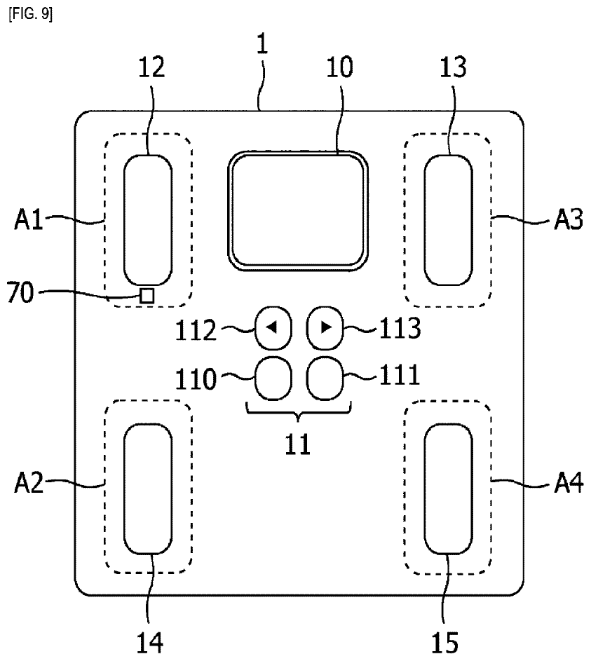

A photoelectric sensor may be disposed on the surface of the biological information measurement device 1, and the amount of light received by the photoelectric sensor may be measured to perform the process of determining whether the subject is barefoot. FIG. 9 is a diagram illustrating an arrangement of a photoelectric sensor. In the example illustrated in FIG. 9, a photoelectric sensor 70 is disposed between the current application electrode 12 and the voltage measurement electrode 14. The first sensor unit 23 may include the photoelectric sensor 70. The photoelectric sensor 70 is preferably disposed near the current application electrode 12 so that the photoelectric sensor 70 reliably comes into contact with the sole of the foot of the subject when the subject gets on the surface of the biological information measurement device 1. For example, the photoelectric sensor 70 may be disposed in the vicinity of the current application electrode 12 so that the photoelectric sensor 70 comes in contact with the pad of the big toe of the subject. The photoelectric sensor 70 may be disposed in a region between a dotted line A1 in FIG. 9 and the current application electrode 12.

The photoelectric sensor 70 may be disposed in the vicinity of the voltage measurement electrode 14. The photoelectric sensor 70 may be disposed in a region between a dotted line A2 in FIG. 9 and the voltage measurement electrode 14. The photoelectric sensor 70 may be disposed between the current application electrode 13 and the voltage measurement electrode 15. The photoelectric sensor 70 may be disposed in the vicinity of the current application electrode 13. The photoelectric sensor 70 may be disposed in a region between a dotted line A3 in FIG. 9 and the current application electrode 13. The photoelectric sensor 70 may be disposed in the vicinity of the voltage measurement electrode 15. The photoelectric sensor 70 may be disposed in a region between a dotted line A4 in FIG. 9 and the voltage measurement electrode 15. A plurality of the photoelectric sensors 70 may be disposed on the surface of the biological information measurement device 1. The fabric of the sock is thin in a rubbed part such as a heel part. In order to avoid erroneous determination, the photoelectric sensor 70 may be prevented from being disposed on the rear side of the surface of the biological information measurement device 1.

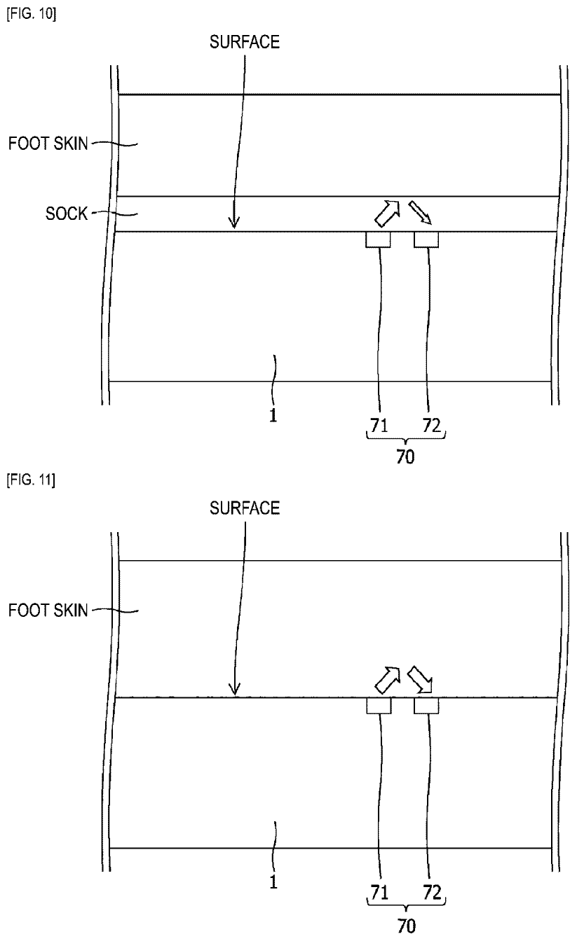

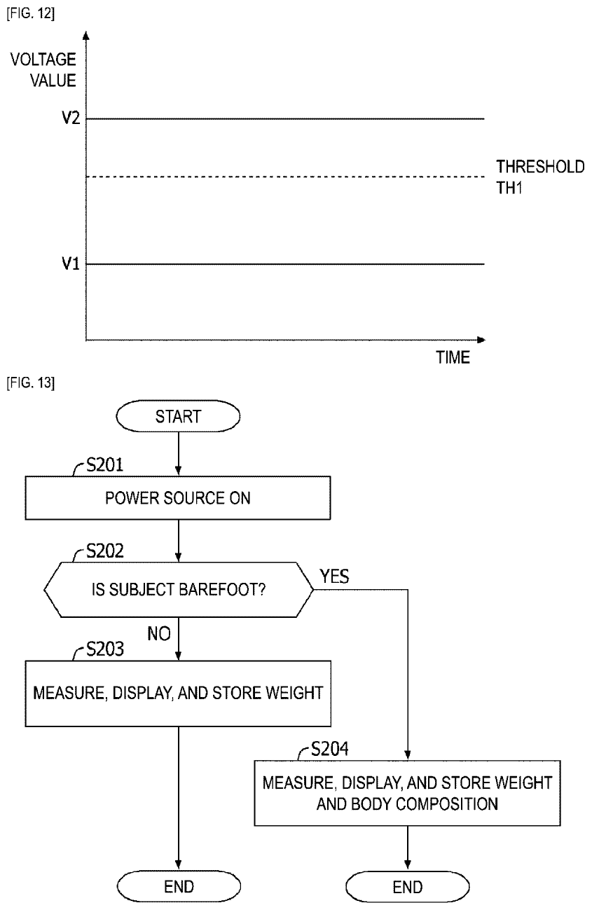

FIG. 10 to FIG. 12 are diagrams illustrating a case where the amount of light received by the photoelectric sensor is measured to determine whether a subject is barefoot. FIG. 10 and FIG. 11 illustrate the biological information measurement device 1 as viewed from the side. The photoelectric sensor 70 includes a light emitting diode (LED) 71 that is a light emitting unit that generates light, and a photodiode 72 that is a light reception unit that receives light. The light emitted by the LED 71 is preferably in a waveband that is not easily absorbed by living body tissues (for example, 500 nm to 1000 nm both inclusive). FIG. 10 illustrates the biological information measurement device 1 in a case where the subject wearing socks is on the surface of the biological information measurement device 1. FIG. 11 illustrates the biological information measurement device 1 in a case where the subject wearing no socks is on the surface of the biological information measurement device 1. FIG. 12 illustrates a value (voltage value) obtained by converting the amount of light received by the photodiode 72 into voltage. The vertical axis in FIG. 12 represents the voltage value and the horizontal axis in FIG. 12 represents the time.

A voltage value V1 illustrated in FIG. 12 is a voltage value obtained with the subject wearing socks being on the surface of the biological information measurement device 1. A voltage value V2 illustrated in FIG. 12 is a voltage value obtained with the subject wearing no socks being on the surface of the biological information measurement device 1. The blood vessels on the body surface are crushed by the soles of the feet of the subject being pressed by the weight. When the subject is wearing no socks, the light emitted from the LED 71 reaches the photodiode 72 with little attenuation. When the subject is wearing socks, the light emitted from the LED 71 is attenuated by the socks and reaches the photodiode 72. Therefore, the voltage value V1 is lower than the voltage value V2.

When the voltage value is equal to or greater than a threshold TH1, the sole state measurement unit 51 determines that the subject is barefoot. When the amount of light received by the photodiode 72 is equal to or greater than the threshold, the sole state measurement unit 51 may determine that the subject is barefoot. The sole state measurement unit 51 outputs a measurement result including a result indicating that the subject is barefoot.

When the voltage value is less than the threshold TH1, the sole state measurement unit 51 determines that the subject is not barefoot. When the amount of light received by the photodiode 72 is less than the threshold, the sole state measurement unit 51 may determine that the subject is not barefoot. The sole state measurement unit 51 outputs a measurement result including a result indicating that the subject is not barefoot.

A plurality of the photoelectric sensors 70 may be disposed on the surface of the biological information measurement device 1. For example, the photoelectric sensor 70 for measuring the state of the sole of the left foot of the subject and the photoelectric sensor 70 for measuring the state of the sole of the right foot of the subject may be disposed on the surface of the biological information measurement device 1.

In step S103, the body motion measurement unit 52 measures the body motion of the subject on the surface of the biological information measurement device 1, based on the measurement value output from at least one of the first sensor unit 23, the second sensor unit 24, or the electrode unit 21, and determines whether the body motion of the subject is large. That is, the body motion measurement unit 52 measures the body motion of the subject on the surface of the biological information measurement device 1, and determines whether the subject is continuously in a stationary state. The stationary state is a state in which variation of the measurement value output from the first sensor unit 23, the second sensor unit 24, or the electrode unit 21 does not reach a predetermined value.

The process of determining whether the body motion of the subject is large will be described. When the foot of the subject on the surface of the biological information measurement device 1 moves, the measurement value output from the first sensor unit 23 varies. The measurement value output from the first sensor unit 23 is, for example, a voltage value or the amount of light received by the photodiode 72. When the measurement value output from the first sensor unit 23 continuously varies for a predetermined period of time, the body motion measurement unit 52 determines that the body motion of the subject is large. The body motion measurement unit 52 may determine that the subject is not continuously in the stationary state, when the measurement value output from the first sensor unit 23 continuously varies for a predetermined period of time. The body motion measurement unit 52 outputs a measurement result including at least one of a result indicating that the body motion of the subject is large or a result indicating that the subject is not continuously in the stationary state. When the measurement value output from the first sensor unit 23 does not continuously vary for the predetermined period of time, the body motion measurement unit 52 determines that the body motion of the subject is small. The body motion measurement unit 52 may determine that the subject is continuously in the stationary state, when the measurement value output from the first sensor unit 23 does not continuously vary for a predetermined period of time. The body motion measurement unit 52 outputs a measurement result including at least one of a result indicating that the body motion of the subject is small or a result indicating that the subject is continuously in the stationary state.

When the subject on the surface of the biological information measurement device 1 makes a predetermined motion, a large measurement value is output from the second sensor unit 24. For example, the predetermined motion is a motion of the subject to see the display unit 10, but is not limited thereto. When the measurement value output from the second sensor unit 24 is continuously equal to or greater than the threshold for a predetermined period of time, the body motion measurement unit 52 determines that the body motion of the subject is large. When the average value of the measurement values output from the second sensor unit 24 within the predetermined period of time is equal to or greater than the threshold, the body motion measurement unit 52 determines that the body motion of the subject is large. In these cases, the body motion measurement unit 52 may determine that the subject is not continuously in the stationary state. The body motion measurement unit 52 outputs a measurement result including at least one of a result indicating that the body motion of the subject is large or a result indicating that the subject is not continuously in the stationary state. When the measurement value output from the second sensor unit 24 is continuously less than the threshold for a predetermined period of time, the body motion measurement unit 52 determines that the body motion of the subject is small. When the average value of the measurement values output from the second sensor unit 24 within the predetermined period of time is less than the threshold, the body motion measurement unit 52 determines that the body motion of the subject is small. In these cases, the body motion measurement unit 52 may determine that the subject is continuously in the stationary state. The body motion measurement unit 52 outputs a measurement result including at least one of a result indicating that the body motion of the subject is small or a result indicating that the subject is continuously in the stationary state. The threshold used for measuring the body motion of the subject may be obtained by an experiment or a simulation. For example, the threshold may be determined based on the measurement value output from the second sensor unit 24 when the subject makes a motion to see the display unit 10.

When the foot of the subject on the surface of the biological information measurement device 1 moves, the measurement value output from the electrode unit 21 varies. The measurement value output from the electrode unit 21 is, for example, a myoelectric. When the measurement value output from the electrode unit 21 continuously varies for a predetermined period of time, the body motion measurement unit 52 determines that the body motion of the subject is large. The body motion measurement unit 52 may determine that the subject is not continuously in the stationary state, when the measurement value output from the electrode unit 21 continuously varies for a predetermined period of time. The body motion measurement unit 52 outputs a measurement result including at least one of a result indicating that the body motion of the subject is large or a result indicating that the subject is not continuously in the stationary state. When the measurement value output from the electrode unit 21 does not continuously vary for the predetermined period of time, the body motion measurement unit 52 determines that the body motion of the subject is small. The body motion measurement unit 52 may determine that the subject is continuously in the stationary state, when the measurement value output from the electrode unit 21 does not continuously vary for a predetermined period of time. The body motion measurement unit 52 outputs a measurement result including at least one of a result indicating that the body motion of the subject is small or a result indicating that the subject is continuously in the stationary state.

When the body motion of the subject is large (step S103; YES), the process proceeds to step S104. When the body motion of the subject is small (step S103; NO), the process proceeds to step S105.

In step S104, the weight measurement unit 53 measures the weight of the subject on the surface of the biological information measurement device 1, based on the measurement value output from the second sensor unit 24. In step S104, the display unit 10 displays information about the weight of the subject. In step S104, the information about the weight of the subject is stored in the storage unit 27. The information about the weight of the subject may be transmitted to the information processing device of the subject via the communication unit 26. The information on the weight of the subject is stored in the storage unit of the information processing device of the subject.

In step S105, the weight measurement unit 53 measures the weight of the subject on the surface of the biological information measurement device 1, based on the measurement value output from the second sensor unit 24. In step S105, the standing position balance measurement unit 55 measures the standing position balance of the subject on the surface of the biological information measurement device 1, based on the measurement value output from the second sensor unit 24. In step S105, the display unit 10 displays information about the weight and standing position balance of the subject. The information on the standing position balance of the subject is, for example, information indicating the position of the center of gravity of the subject is a standing position, but is not limited thereto. In step S105, the information about the weight and the standing position balance of the subject is stored in the storage unit 27. The information about the weight and the standing position balance of the subject may be transmitted to the information processing device of the subject via the communication unit 26. The information on the weight and the standing position balance of the subject is stored in the storage unit of the information processing device of the subject.

In step S106, the body motion measurement unit 52 measures the body motion of the subject on the surface of the biological information measurement device 1, based on the measurement value output from at least one of the first sensor unit 23, the second sensor unit 24, or the electrode unit 21, and determines whether the body motion of the subject is large.

When the body motion of the subject is large (step S106; YES), the process proceeds to step S107. When the body motion of the subject is small (step S106; NO), the process proceeds to step S108.

In step S107, the weight measurement unit 53 measures the weight of the subject on the surface of the biological information measurement device 1, based on the measurement value output from the second sensor unit 24. In step S107, the body composition measurement unit 54 measures the body composition of the subject on the surface of the biological information measurement device 1, based on the bioelectrical impedance output from the impedance measurement unit 22 and the weight of the subject. In step S107, the display unit 10 displays information about the weight and the body composition of the subject.

In step S107, the information on the weight and the body composition of the subject is stored in the storage unit 27. The information on the weight and the body composition of the subject may be transmitted to the information processing device of the subject via the communication unit 26. The information on the weight and the body composition of the subject is stored in the storage unit of the information processing device of the subject.

In step S108, the weight measurement unit 53 measures the weight of the subject on the surface of the biological information measurement device 1, based on the measurement value output from the second sensor unit 24. In step S108, the body composition measurement unit 54 measures the body composition of the subject on the surface of the biological information measurement device 1, based on the bioelectrical impedance output from the impedance measurement unit 22 and the weight of the subject. In step S108, the standing position balance measurement unit 55 measures the standing position balance of the subject on the surface of the biological information measurement device 1, based on the measurement value output from the second sensor unit 24. In step S108, the cardiovascular measurement unit 56 measures the cardiovascular state of the subject on the surface of the biological information measurement device 1, based on the measurement value output from the second sensor unit 24 and the bioelectrical impedance output from the impedance measurement unit 22. In step S108, the display unit 10 displays information about the weight, the body composition, the standing position balance, and the cardiovascular state of the subject. The information about the cardiovascular state is a cardiovascular indicator, but is not limited thereto.

In step S108, the information about the weight, the body composition, the standing position balance, and the cardiovascular state of the subject is stored in the storage unit 27. The information about the weight, the body composition, the standing position balance, and the cardiovascular state of the subject may be transmitted to the information processing device of the subject via the communication unit 26. The Information about the weight, the body composition, the standing position balance, and the cardiovascular state of the subject is stored in the storage unit of the information processing device of the subject.

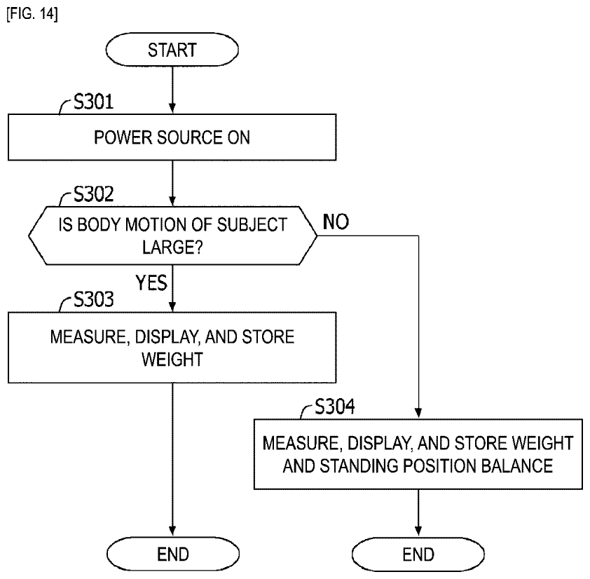

A second operation of the biological information measurement device 1 will be described with reference to FIG. 13. FIG. 13 is a flowchart illustrating the second operation of the biological information measurement device 1. For example, when a second operation mode is set for the biological information measurement device 1, the control unit 20 reads out the program stored in the storage unit 27, and thereby, each process in the flowchart of FIG. 13 is executed.

In step S201, the subject presses the switch 110 of the biological information measurement device 1 to turn on the power of the biological information measurement device 1. The power of the biological information measurement device 1 may be turned on when the subject is placed on the surface of the biological information measurement device 1 in step S201.

In step S202, the sole state measurement unit 51 measures the state of the sole of the foot of the subject placed on the surface of the biological information measurement device 1, based on a measurement value output from at least one of the electrode unit 21 or the first sensor unit 23, and whether the subject is barefoot is determined. When the subject is not barefoot (step S202; NO), the process proceeds to step S203. When the subject is barefoot (step S202; YES), the process proceeds to step S204.

In step S203, the weight measurement unit 53 measures the weight of the subject on the surface of the biological information measurement device 1, based on the measurement value output from the second sensor unit 24. In step S203, the display unit 10 displays information about the weight of the subject. In step S203, the information about the weight of the subject is stored in the storage unit 27. The information about the weight of the subject may be transmitted to the information processing device of the subject via the communication unit 26. The information on the weight of the subject is stored in the storage unit of the information processing device of the subject.

In step S204, the weight measurement unit 53 measures the weight of the subject on the surface of the biological information measurement device 1, based on the measurement value output from the second sensor unit 24. In step S204, the body composition measurement unit 54 measures the body composition of the subject on the surface of the biological information measurement device 1, based on the bioelectrical impedance output from the impedance measurement unit 22 and the weight of the subject. In step S204, the display unit 10 displays information about the weight and the body composition of the subject. In step S204, the information on the weight and the body composition of the subject is stored in the storage unit 27. The information on the weight and the body composition of the subject may be transmitted to the information processing device of the subject via the communication unit 26. The information on the weight and the body composition of the subject is stored in the storage unit of the information processing device of the subject.

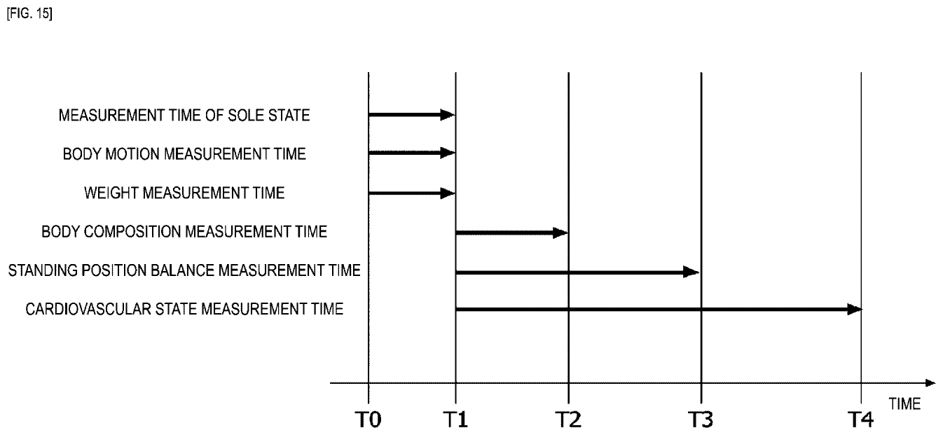

A third operation of the biological information measurement device 1 will be described with reference to FIG. 14. FIG. 14 is a flowchart illustrating the third operation of the biological information measurement device 1. For example, when a third operation mode is set for the biological information measurement device 1, the control unit 20 reads out the program stored in the storage unit 27, and thereby, each process in the flowchart of FIG. 14 is executed.

In step S301, the subject presses the switch 110 of the biological information measurement device 1 to turn on the power of the biological information measurement device 1. The power of the biological information measurement device 1 may be turned on when the subject is placed on the surface of the biological information measurement device 1 in step S301.

In step S302, the body motion measurement unit 52 measures the state of the body motion of the subject on the surface of the biological information measurement device 1, based on the measurement value output from at least one of the first sensor unit 23, the second sensor unit 24, or the electrode unit 21, and determines whether the body motion of the subject is large.

When the body motion of the subject is large (step S302; YES), the process proceeds to step S303. When the body motion of the subject is small (step S302; NO), the process proceeds to step S304.

In step S303, the weight measurement unit 53 measures the weight of the subject on the surface of the biological information measurement device 1, based on the measurement value output from the second sensor unit 24. In step S303, the display unit 10 displays information about the weight of the subject. In step S303, the information about the weight of the subject is stored in the storage unit 27. The information about the weight of the subject may be transmitted to the information processing device of the subject via the communication unit 26. The information on the weight of the subject is stored in the storage unit of the information processing device of the subject.

In step S304, the weight measurement unit 53 measures the weight of the subject on the surface of the biological information measurement device 1, based on the measurement value output from the second sensor unit 24. In step S304, the standing position balance measurement unit 55 measures the standing position balance of the subject on the surface of the biological information measurement device 1, based on the measurement value output from the second sensor unit 24. In step S304, the display unit 10 displays information about the weight and standing position balance of the subject. In step S304, the information about the weight and the standing position balance of the subject is stored in the storage unit 27. The information about the weight and the standing position balance of the subject may be transmitted to the information processing device of the subject via the communication unit 26. The information on the weight and the standing position balance of the subject is stored in the storage unit of the information processing device of the subject.

FIG. 15 is a time chart of a process of measuring a sole state, a process of measuring a body motion, a process of measuring a weight, a process of measuring a body composition, a process of measuring a standing position balance, and a process of measuring a cardiovascular state. The time period for measuring the state of the sole is an elapsed time period from a timing (T0) at which the subject gets on the surface of the biological information measurement device 1 to a timing (T1) at which the process of measuring the state of the sole ends. The time period for measuring the body motion is an elapsed time period from the timing (T0) at which the subject gets on the surface of the biological information measurement device 1 to the timing (T1) at which the process of measuring the body motion ends. The time period for measuring the weight is an elapsed time period from the timing (T0) at which the subject gets on the surface of the biological information measurement device 1 to the timing (T1) at which the process of measuring the weight ends. The time period for measuring the body composition is an elapsed time period from the timing (T1) to a timing (T2) at which the process of measuring the body composition ends. The time period for measuring the standing position balance is an elapsed time period from the timing (T1) to a timing (T3) at which the process of measuring the standing position balance ends. The time period for measuring the cardiovascular state is an elapsed time period from the timing (T1) to a timing (T4) at which the process of measuring the cardiovascular state ends.

The first measurement unit 41 includes the sole state measurement unit 51 and the body motion measurement unit 52. The first measurement unit 41 performs at least one of the first measurement process of measuring the state of the sole of the foot of the subject on the surface of the biological information measurement device 1 or the second measurement process of measuring the body motion of the subject on the surface of the biological information measurement device 1. When the first operation mode is set for the biological information measurement device 1, the first measurement unit 41 performs both the first measurement process and the second measurement process, and outputs the measurement result of the first measurement process and the measurement result of the second measurement process. When the second operation mode is set for the biological information measurement device 1, the first measurement unit 41 performs the first measurement process and outputs the measurement result of the first measurement process. When the third operation mode is set for the biological information measurement device 1, the first measurement unit 41 performs the second measurement process and outputs the measurement result of the second measurement process.

The second measurement unit 42 includes the weight measurement unit 53, the body composition measurement unit 54, the standing position balance measurement unit 55, and the cardiovascular measurement unit 56. The second measurement unit 42 selects at least one measurement item from the plurality of measurement items related to the living body of the subject, based on at least one of the measurement result of the first measurement process or the measurement result of the second measurement process, and measures the biological information of the subject. The plurality of measurement items related to the living body of the subject include a measurement item for measuring the weight, a measurement item for measuring the body composition, a measurement item for measuring the standing position balance, and a measurement item for measuring the cardiovascular state. According to the biological information measurement device 1, at least one measurement item is selected from the plurality of measurement items related to the living body of the subject according to the state of the sole of the foot of the subject and the body motion of the subject, and the biological information of the subject is measured, and thus, usability in the measurement of the biological information is improved. In a known body composition meter, for example, even when only the body weight is to be measured, the subject needs to be on the body composition meter with his/her bare feet. Therefore, according to the known body composition meter, it is necessary to wait until the measurement of the measurement items other than the weight of the subject is completed, and thus the usability in the measurement of the biological information is compromised.

A sensor may be used as both a sensor for measuring the body motion of the subject on the surface of the biological information measurement device 1 and a sensor for measuring the weight of the subject on the surface of the biological information measurement device 1. For example, the body motion of the subject on the surface of the biological information measurement device 1 may be measured and the weight of the subject on the surface of the biological information measurement device 1 may be measured, using the plurality of load cells of the second sensor unit 24. By using a sensor serving as both the sensor for measuring the body motion of the subject on the surface of the biological information measurement device 1 and the sensor for measuring the weight of the subject on the surface of the biological information measurement device 1, cost reduction is possible.

The plurality of measurement items related to the living body of the subject may include a measurement item that is measured using a sensor that measures body motion of the subject on the surface of the biological information measurement device 1. The sensor that measures the body motion of the subject on the surface of the biological information measurement device 1 is, for example, the plurality of load cells of the second sensor unit 24. The items for which measurement is performed using the plurality of load cells of the second sensor unit 24 are the measurement item for measuring the weight, the measurement item for measuring the standing position balance, and the measurement item for measuring the cardiovascular state.

A case where the first operation mode is set for the biological information measurement device 1 will be described. Here, a result indicating that the subject is not barefoot is referred to as a result (A), and a result indicating that the subject is barefoot is referred to as a result (B). A result indicating that the body motion of the subject is large or a result indicating that the subject is continuously in the stationary state is referred to as a result (C). A result indicating that the body motion of the subject is small or a result indicating that the subject is continuously in the stationary state is referred to as a result (D).

For example, when the subject is wearing socks and thus the subject is not barefoot, the measurement accuracy of the bioelectrical impedance is compromised. Thus, it is not preferable to measure the body composition and the cardiovascular state of the subject in such a case. It is not preferable to measure the standing position balance without the subject being stationary. In order to measure the cardiovascular state of the subject, the subject needs to be continuously in the stationary state for 20 to 30 seconds. Therefore, when the measurement result of the first measurement process includes the result (A) and the measurement result of the first measurement process includes the result (C), the second measurement unit 42 selects the measurement item for measuring the weight from the plurality of measurement items related to the living body of the subject, and measures the biological information of the subject. Since only the weight of the subject is measured and no measurement is performed for the other measurement items, the time from the start to the completion of the measurement of the biological information of the subject is shortened. Specifically, since sensor output, algorithm processing, display and storage processing for the biological information, and the like are omitted from the measurement of the body composition, the standing position balance, and the cardiovascular state of the subject, the time period from the start to the completion of the measurement of the biological information of the subject is shortened. When the body composition or the cardiovascular state of the subject wearing socks is measured using a known body composition meter, an error occurs in the middle of measurement, making it impossible to measure the body composition or the cardiovascular state of the subject, for example. In this case, with the known body composition meter, since a process such as error display is performed, the time period from the start to the completion of the measurement of the biological information of the subject becomes long.

When the measurement result of the first measurement process includes the result (B) and the measurement result of the first measurement process includes the result (C), the second measurement unit 42 selects at least one measurement item that can be measured in a state where the subject is not stationary from the plurality of measurement items related to the living body of the subject, and measures the biological information of the subject. The measurement item that can be measured in a state where the subject is not stationary is a measurement item for measuring the weight and a measurement item for measuring the body composition. Since the weight and the body composition of the subject are measured and no measurement is performed for the other measurement items, the time from the start to the completion of the measurement of the biological information of the subject is shortened. Specifically, since sensor output, algorithm processing, display and storage processing for the biological information, and the like are omitted from the measurement of the standing position balance and the cardiovascular state of the subject, the time period from the start to the completion of the measurement of the biological information of the subject is shortened.

When the measurement result of the first measurement process includes the result (A) and the measurement result of the first measurement process includes the result (D), the second measurement unit 42 selects at least one measurement item that can be measured without using the measurement result of the bioelectrical impedance from the plurality of measurement items related to the living body of the subject, and measures the biological information of the subject. The measurement items that can be measured without using the bioelectrical impedance measurement result are the measurement item for measuring the weight and the measurement item for measuring the standing position balance. Since the weight and the standing position balance of the subject are measured and no measurement is performed for the other measurement items, the time from the start to the completion of the measurement of the biological information of the subject is shortened. Specifically, since sensor output, algorithm processing, display and storage processing for the biological information, and the like are omitted from the measurement of the body composition and the cardiovascular state of the subject, the time period from the start to the completion of the measurement of the biological information of the subject is shortened.

When the measurement result of the first measurement process includes the result (B) and the measurement result of the first measurement process includes the result (D), the second measurement unit 42 selects all of the plurality of measurement items related to the living body of the subject and measures the biological information of the subject. When the barefoot subject is on the surface of the biological information measurement device 1 and the subject is continuously in the stationary state, the biological information of the subject is measured with the subject regarded as having an intention to measure the weight, the body composition, the standing position balance, and the cardiovascular state.

In a case where the measurement of the biological information of the subject is performed for all of the plurality of measurement items related to the living body of the subject, the display unit 10 may not display the information while the measurement process is being performed by the second measurement unit 42. The motion of the subject to see the information displayed on the display unit 10 (the motion of seeing the feet) affects the measurement of the cardiovascular state. The display unit 10 does not display information while the measurement process is performed by the second measurement unit 42, and thus it is possible to suppress the body motion of the subject, and the accuracy of measurement on the cardiovascular state is improved.

When the sole of the foot of the subject placed on the surface of the biological information measurement device 1 is in a low temperature state, the measurement accuracy of the state of the sole of the foot of the subject using the plurality of electrodes is compromised, and the subject may be erroneously determined not to be barefoot although the subject is barefoot. Therefore, when the temperature of the sole of the foot of the subject on the surface of the biological information measurement device 1 is equal to or lower than a predetermined temperature, the first measurement unit 41 may perform the second measurement process and output the measurement result of the second measurement process. In this case, the second measurement unit 42 selects at least one measurement item that can be measured without using the measurement result of the bioelectrical impedance from the plurality of measurement items related to the living body of the subject, based on the measurement result of the second measurement process, and measures the biological information of the subject. Thus, when the temperature of the sole of the foot of the subject placed on the surface of the biological information measurement device 1 is equal to or lower than the predetermined temperature, the first measurement unit 41 does not perform the first measurement process, and thus it is possible to avoid the erroneous determination. The display unit 10 may display a message “Body composition and the cardiovascular state cannot be measured because the sole of the foot is cold”.

In a case where the subject gets off the biological information measurement device 1 before completion of the measurement of the biological information for some of the measurement items selected from the plurality of measurement items related to the living body of the subject, the measurement result of the biological information that has been measured at the time point when the subject has gotten off the biological information measurement device 1 may be displayed on the display unit 10. For example, when the subject gets off the biological information measurement device 1 between the timing (T2) and the timing (T3) in FIG. 15, the display unit 10 displays the measurement results of the biological information measured between the start of the measurement of the biological information and the timing (T2) in FIG. 15. For example, when the subject gets off the biological information measurement device 1 between the timing (T3) and the timing (T4) in FIG. 15, the display unit 10 displays the measurement results of the biological information measured between the start of the measurement of the biological information at the timing (T3) in FIG. 15.

### Computer Readable Recording Medium

A program or recording medium for causing an information processing device, other machines and apparatuses (hereinafter, referred to as a computer or the like) to realize any of the functions described above can be recorded on a recording medium readable by the computer and the like. Then, the computer or the like is caused to load the program onto a recording medium and execute the program, thereby providing the functions.

Here, the recording medium readable by the computer or the like refers to a recording medium that can store information such as data and programs by an electrical, magnetic, optical, mechanical, or chemical action and can be read from the computer or the like. Among such recording media, those removable from the computer or the like include, for example, a flexible disk, a magneto-optical disk, a CD-ROM, a CD-R/W, a DVD, a Blu-ray disk, a flash memory, and the like. Further, such a recording medium fixed to the computer or the like includes a hard disk, a ROM, and the like.

### REFERENCE SIGNS LIST

1: Biological information measurement device
10: Display unit
11: Operation unit
12, 13: Current application electrode
14, 15: Voltage measurement electrode
20: Control unit
21: Electrode unit
22: Impedance measurement unit
23: First sensor unit
24: Second sensor unit
25: Temperature measurement unit
26: Communication unit
27: Storage unit
31, 32, 33, 34: Load cell
35: Acceleration sensor
41: First measurement unit
42: Second measurement unit
51: Sole state measurement unit
52: Body motion measurement unit
53: Weight measurement unit
54: Body composition measurement unit
55: Standing position balance measurement unit
56: Cardiovascular measurement unit
60: Auxiliary electrode

1. A biological information measurement device with a placement surface configured to allow placement of a subject, the biological information measurement device comprising:
a first measurement unit configured to perform at least one of a first measurement process of measuring a state of a sole of a foot of the subject placed on the placement surface or a second measurement process of measuring a body motion of the subject placed on the placement surface; and
a second measurement unit configured to, based on at least one of a measurement result of the first measurement process or a measurement result of the second measurement process, select at least one measurement item from a plurality of measurement items related to a living body of the subject and measure biological information of the subject.

2. The biological information measurement device according to claim 1, wherein
the plurality of measurement items include a measurement item for measuring a weight of the subject placed on the placement surface, and
a sensor configured to measure the body motion of the subject placed on the placement surface and a sensor configured to measure the weight of the subject placed on the placement surface are configured as a common sensor.

3. The biological information measurement device according to claim 1, wherein
the plurality of measurement items include a measurement item that is measured using a sensor configured to measure the body motion of the subject placed on the placement surface.

4. The biological information measurement device according to claim 2, wherein
the plurality of measurement items include a measurement item that is measured using a sensor configured to measure the body motion of the subject placed on the placement surface.

5. The biological information measurement device according to claim 1, wherein
the first measurement unit performs the first measurement process and the second measurement process, and
when the measurement result of the first measurement process includes a result indicating that the subject is not barefoot and includes a result indicating interruption of a stationary state of the subject, the second measurement unit selects a measurement item for measuring a weight from the plurality of measurement items and measures the biological information of the subject.

6. The biological information measurement device according to claim 1, wherein
the first measurement unit performs the first measurement process and the second measurement process, and
when the measurement result of the first measurement process includes a result indicating that the subject is barefoot and includes a result indicating interruption of a stationary state of the subject, the second measurement unit selects at least one measurement item that is measurable in a state where the subject is not still from the plurality of measurement items and measures the biological information of the subject.

7. The biological information measurement device according to claim 1, wherein
the first measurement unit performs the first measurement process and the second measurement process, and
when the measurement result of the first measurement process includes a result indicating that the subject is not barefoot and includes a result indicating that the subject is continuously in a stationary state, the second measurement unit selects at least one measurement item that is measurable without using a bioelectrical impedance measurement result from the plurality of measurement items and measures the biological information of the subject.

8. The biological information measurement device according to claim 1, wherein
the first measurement unit performs the first measurement process and the second measurement process, and
when the measurement result of the first measurement process includes a result indicating that the subject is barefoot and the measurement result of the second measurement process includes a result indicating that the subject is continuously in a stationary state, the second measurement unit selects all of the plurality of measurement items and measures the biological information of the subject.

9. The biological information measurement device according to claim 8 comprising a display unit configured to display information, wherein
the display unit does not display the information while a measurement process is performed by the second measurement unit.

10. The biological information measurement device according to claim 1 comprising a temperature measurement unit configured to measure a temperature of the sole of the foot of the subject placed on the placement surface, wherein
when the temperature of the foot of the subject is equal to or lower than a predetermined temperature, the first measurement unit performs the second measurement process, and
the second measurement unit selects at least one measurement item that is measurable without using a bioelectrical impedance measurement result from the plurality of measurement items and measures the biological information of the subject, based on the measurement result of the second measurement process.

11. A biological information measurement method executed by a computer of a biological information measurement device with a placement surface configured to allow placement of a subject, the biological information measurement method comprising:
performing at least one of a first measurement process of measuring a state of a sole of a foot of the subject placed on the placement surface or a second measurement process of measuring a body motion of the subject placed on the placement surface; and
based on at least one of a measurement result of the first measurement process or a measurement result of the second measurement process, selecting at least one measurement item from a plurality of measurement items related to a living body of the subject and measuring biological information of the subject.

12. A recording medium for causing a computer of a biological information measurement device with a placement surface configured to allow placement of a subject to execute:
performing at least one of a first measurement process of measuring a state of a sole of a foot of the subject placed on the placement surface or a second measurement process of measuring a body motion of the subject placed on the placement surface; and
based on at least one of a measurement result of the first measurement process or a measurement result of the second measurement process, selecting at least one measurement item from a plurality of measurement items related to a living body of the subject and measuring biological information of the subject.
